(19) United States
(12) Patent Application Publication
(10) Pub. No.: US 2025/0182868 A1
(43) Pub. Date: Jun. 5, 2025

(54) EMERGENCY CONTACT SYSTEM FOR ACCESSING MEDICAL AND PERSONAL IDENTIFIABLE INFORMATION

(71) Applicant: Securus Documents, LLC, Carrollton, VA (US)

(72) Inventors: John M. Gomery, Carrollton, VA (US); Terry J. Johnson, Carrollton, VA (US)

(21) Appl. No.: 18/657,281

(22) Filed: May 7, 2024

Related U.S. Application Data
(60) Provisional application No. 63/605,084, filed on Dec. 1, 2023.

(51) Int. Cl. G16H 10/65; G06K 19/06; G16H 40/67; G16H 80/00

(52) U.S. Cl. CPC G16H 10/65; G06K 19/06009; G16H 40/67; G16H 80/00

(57) ABSTRACT

A system for the on-demand and automated transfer of medical information to on-site medical care providers is disclosed. The system includes a computing device in operable connection with a network and an application server in operable communication with the network to host an application program for the automated transfer of medical information. The application program includes a user interface module for providing access to the medical information by a medical care provider on-site. A QR code provided on a contact card, mobile device or other means for displaying a QR code may be scanned by the care provider to initiate an identity verification process and determine if the care provider is HIPAA certified. The HIPAA compliant information is then accessed by the care provider to aid in quality of care.

### CROSS-REFERENCE TO RELATED APPLICATIONS

The present application claims priority to U.S. Provisional Application No. 63/605,084 filed Dec. 1, 2023, titled “EMERGENCY CONTACT SYSTEM FOR ACCESSING MEDICAL AND PERSONAL IDENTIFIABLE INFORMATION,” which is hereby incorporated by reference in its entirety.

### TECHNICAL FIELD

The embodiments generally relate to computerized systems for accessing personal information, and more particularly to the on-demand and automated transfer of medical information.

### BACKGROUND

Advances in computing technologies have led to many innovations in data storage and processing, record keeping, and the transmission of data in general. Fields, such as medicine, have particularly benefited in these advances, as medical information is stored and transmitted using network communications. However, first responders are often not equipped to readily access a patient's information as the patient may be unidentifiable, incapacitated, or otherwise unable to provide information to the emergency medical personnel on-site at the time. Accessing accurate patient information in an efficient manner may provide emergency personnel with the information needed to perform the highest quality of care.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments described herein relate to a system for the on-demand and automated transfer of medical information to on-site medical care providers. The system includes a computing device in operable connection with a network and an application server in operable communication with the network to host an application program for the automated transfer of medical information. The application program includes a user interface module for providing access to the medical information by a medical care provider on-site. A QR code provided on a contact card, mobile phone, or other means of displaying the QR code such that it may be scanned by the care provider to initiate an identity verification process and determine if the care provider is HIPAA certified. The HIPAA compliant information is then accessed by the care provider to aid in quality of care.

During the identity verification process, if it is determined that the medical care provider is HIPAA certified, information is transmitted to the medical care provider. In the instance where the medical care provider is not HIPAA certified, the system displays a selectable webpage to which the user is directed. The user then utilizes the webpage to enter information which may be sent to a deputy/agent for medical and legal responses. In such, the transmission and storage of the patient's medical information remains HIPAA compliant.

In one aspect, the digital emergency contact card is also provided as a physical emergency contact card. The physical emergency contact card may be carried by the patient, such that it can be accessed when needed. The physical contact card may include the scannable code displayed on the surface of the emergency contact card.

In one aspect, the scannable code may be a QR code. Further, the scannable code may also be provided as an account number which is displayed on the emergency contact card.

In one aspect, a verification module is used to receive an input from the user to indicate if the user is a patient, a care provider, or a deputy agent. The verification module may then verify the identity of the patient, the care provider, or the deputy agent using the identifiable information they provide.

In one aspect, a message module facilitates the automated transmission of at least one message to a deputy agent. This message is transmitted automatically once the emergency contact card is scanned by the care provider. The message module may also facilitate the automated transmission of the patient-associated information to the care provider if this feature has been enabled by the patient.

In one aspect, a user module permits the association of a user account with a plurality of user permissions. This is determined by the verification module once the user indicates their user-type (e.g., a patient, a care provider, or a deputy agent).

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computing system, according to some embodiments;

FIG. 2 illustrates a block diagram of the application program in operable communication with the computing system, according to some embodiments;

FIG. 3 illustrates a block diagram of the databases in communication with the application program;

FIG. 4A illustrates a flowchart of a method for providing an emergency contact system for accessing medical and personal identifiable information as it is related to the patient, according to some embodiments;

FIG. 4B illustrates a flowchart of a method for providing an emergency contact system for accessing medical and personal identifiable information as it is related to the EMT, according to some embodiments;

FIG. 4C illustrates a flowchart of a method for providing an emergency contact system for accessing medical and personal identifiable information as it is related to care provider, according to some embodiments;

FIG. 5 illustrates a top plan view of an emergency contact card, according to some embodiments; and

FIG. 6 illustrates a screenshot of the digital emergency contact card user interface, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

In general, the embodiments provided herein relate to a system for the on-demand and automated transfer of medical information to emergency personnel, first responders, and other care providers. The system includes a contact card which includes a QR code or similar implement to be scanned by the care provider. Once a third-party identity verification process is completed, the scanning of the QR code transmits medical information to the medical personnel. The system automates the transfer of personal identifiable information (including the patient's name, age, date of birth, social security number, and other relevant information) which can be utilized by the medical care provider at the scene.

As used herein, the term “user” may relate to anyone having access to the system including a patient who is associated with the emergency contact card, emergency medical service (EMS) personnel, hospital staff, a deputy agent, or other persons involved in the care of a patient.

As used herein, the term “care provider” is used to collectively refer to any verified user of the system who has been authorized to scan a patient emergency contact card and access patient-related information associated with the emergency contact card. This can include EMS personnel, hospital personnel, and other care providers.

As used herein, the term “deputy agent” refers to an individual who has been given authorization to act on behalf of the patient. The deputy agent may not necessarily be a registered user of the system but may be contacted by the care provider if the patient has provided the contact information for the deputy agent. In such, the deputy agent may receive automated communications from the system if/when the patient's emergency contact card is scanned by the care provider.

The use of a contact card is one example of how the QR code may be displayed. However, it is contemplated that the QR code may be displayed on a mobile device or other means for displaying a QR code. The contact card is used as a non-limiting example herein.

In some embodiments, the third-party identity verification process may determine if the medical care provider is Health Insurance Portability and Accountability Act (HIPAA) certified. If the medical care provider is HIPAA certified, information is transmitted to the medical care provider. In the instance where the medical care provider is not HIPAA certified, the system contacts a deputy/agent for medical and legal responses. In such, the transmission and storage of the patient's medical information remains HIPAA compliant.

In some embodiments, the data transmitted through the system is protected in a HIPAA compliant manner, thus protecting the data from ransomware attacks or similar malicious activities.

In some embodiments, once the EMS personnel transport the patient to the hospital, the medical staff at the hospital may utilize the emergency contact card to make contact with a deputy agent appointed by the user (the patient) who may act on their behalf In some cases, the deputy agent may have an executed power of attorney which is helpful for the hospital staff when finding a direction of care for the patient.

In some embodiments, the system provides messaging capabilities in real-time. This allows for users to transmit messages to contacts stored in the emergency contact card database, including a deputy agent.

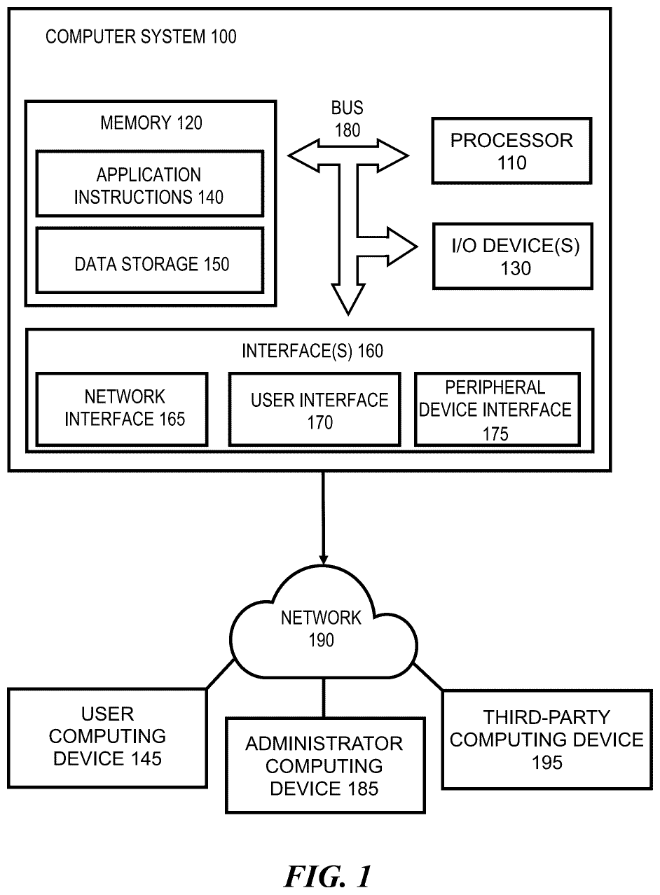

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized to establish credentials, create a user profile, and otherwise interact with the features of the system. The third-party computing device 195 may be utilized by third parties to receive communications from the user computing device and/or administrative computing device 185.

In some embodiments, the third-party computing device may be the computing device of a deputy agent. In such, the system can send automated messages, emails, notifications, and/or alerts to the deputy agent in the event that the patient's emergency contact card has been scanned by a care provider.

In some embodiments, the third-party computing device may be the computing device of a care provider. In such, the third-party computing device is utilized by the care provider to scan the emergency contact card of the patient and access the various functionalities described herein.

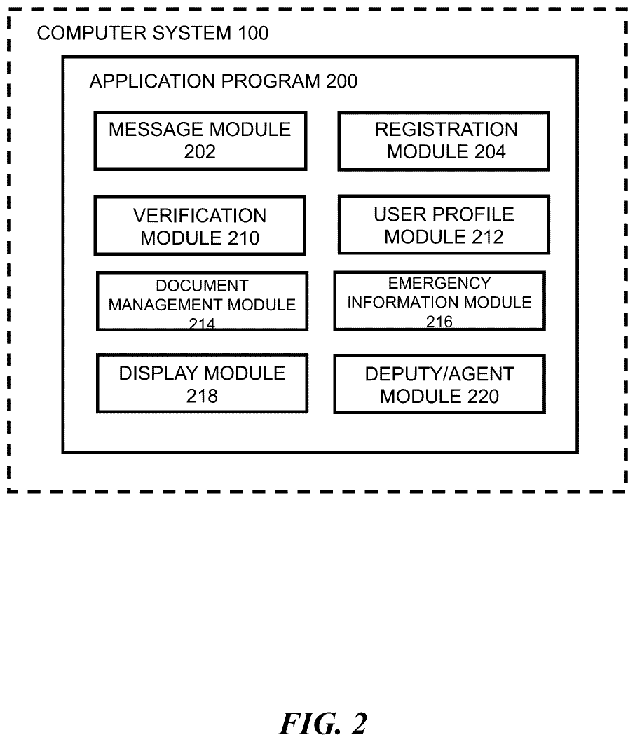

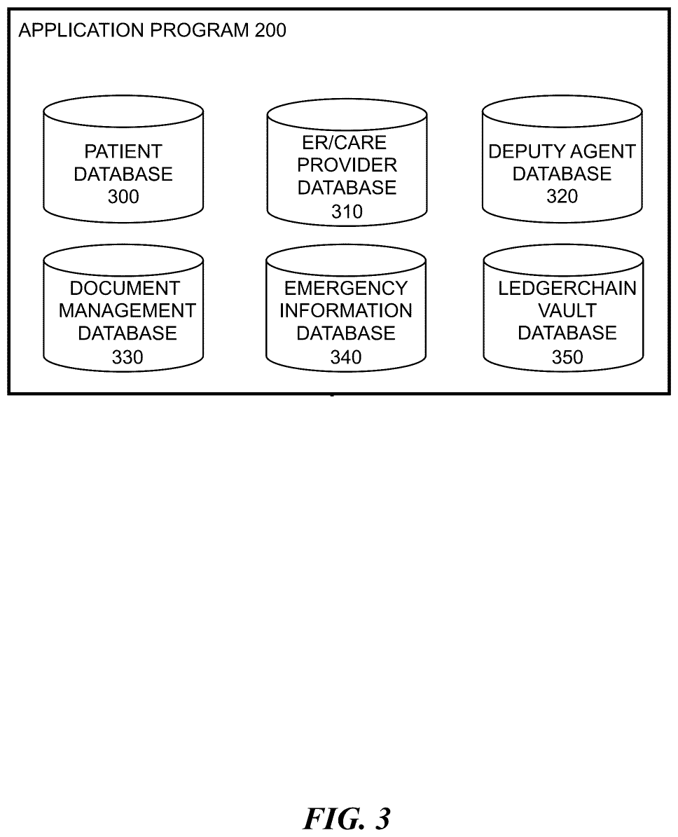

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases (see FIG. 3). In particular, FIG. 2 is a block diagram showing the modules and engines needed to perform specific tasks within the application program 200. FIG. 3 illustrates a block diagram of the databases in communication with the computer system 100 and application program 200 displayed in FIG. 2.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a message module 202, a registration module 204, a verification module 210, a user profile module 212, a document management module 214, an emergency information module 216, a display module 218, and a Deputy/Agent module 220.

In some embodiments, the message module 202 is configured for receiving, processing, and transmitting a user command and/or one or more data streams. In such embodiments, the message module 202 performs communication functions between various devices, including the user computing device 145, the administrator computing device 185, and a third-party computing device 195. In some embodiments, the message module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

The message module 202 may enable, for example, the communication between users. The message module 202 allows for the fully automated communication and transmittal of notification and/or alerts between users. For example, the message module 202 may facilitate the automated communication between the system and a deputy agent. In this example, once an EMT (or other EMS personnel) scans the emergency contact card, the system will automatically transmit a notification to the deputy agent associated with the patient. This provides a fully automated means of notifying the deputy agent of the patient's status.

In some embodiments, the message module 202 may facilitate the automated transmission of communications related to the user's medical information. For example, the message module 202 may communicate to the EMT that the user has given approval to receive one or more specific treatments. The medical information may include triage medical information, advanced healthcare directive(s), the user's contact and personal information (e.g., name, date-of-birth, social security number, address, phone number, email, account information, etc.).

In some embodiments, the message module 202 may transmit a message, notification, and/or alert to EMS personnel if the user has not provided permission to share their medical information, personnel information or other user-associated information. If the EMS personnel receives this communication, the deputy agent may still be automatically contacted using the message module 202.

In some embodiments, the message module 202 allows the deputy agent to transmit a power of attorney, medical information, contact information, insurance information, and other useful information to the care provider.

In some embodiments, a database engine is configured to facilitate the storage, management, and retrieval of data to and from one or more storage mediums, such as the one or more internal databases described herein. In some embodiments, the database engine is coupled to an external storage system. In some embodiments, the database engine is configured to apply changes to one or more databases. In some embodiments, the database engine comprises a search engine component for searching through thousands of data sources stored in different locations.

In some embodiments, the database engine is operable to communicate with the databases illustrated in FIG. 3 to transmit and/or receive information stored in the databases.

In some embodiments, the registration module 204 is utilized by users to create an account and register with the system. This allows for the user to create a user profile, using the user profile module.

In some embodiments, the verification module 210 is operable to verify the identities of the users of the system. In some embodiments, the verification module 210 is in communication with one or more third-party systems which aid in user identification and verification. The verification module 210 may first verify new users of the system utilizing a third-party verification system. The verification process involves the steps of the user inputting the personal information which may include the input of images of the user. The images may also include an image of an identity card, such as a government issued identity card, driver's license, passport, etc. The user-associated information is stored in a user database to associate the user-associated information with a user account and an emergency contact card.

In some embodiments, the verification module 210 may be utilized to verify the identity of the EMS personnel (or other care provider). During the EMS personnel verification process, the EMS personnel registers with the system and are provided with an authorization token by their employer. The EMS personnel may be verified by a third-party system. Once verified, the EMS personnel may utilize the application program to scan the patient's emergency contact card. The verification module 210 verifies that the EMS personnel or other care provided is an authorized user and the patient's deputy agent is contacted automatically once the verification process is complete.

In some embodiments, the verification module 210 may verify and authorize a hospital or other care facility to access the system and user information. Once the patient arrives at the care facility, a care provider at the care facility may scan the patient's emergency contact card, thus granting them access to the patient's information. This may also automatically transmit a message to the deputy agent associated with the patient, notifying them that the patient is at the care facility.

In some embodiments, the user profile module 212 facilitates the creation of a user account. This can include an account associated with an emergency contact card (i.e., the patient account). The user account module 212 also facilitates the creation of accounts associated with care providers (e.g., EMS personnel, hospital personnel, deputy agents, and/or healthcare facilities). The user module 212 may control user permissions, such as allowing care providers to scan the emergency contact card to receive medical data. This feature is not allowed for patient accounts, who use the system to upload medical information and identify deputy agents to which they are associated. In such, the user module 212 is provided with secure protocols to ensure the secure transmission and storage of information within the system.

In some embodiments, the document management module 214 is operable to receive documents associated with users of the system. The document management module 214 may be in operable communication with the verification module 210 to provide documents useful in user verification processes described herein. The document management module 214 is in communication with a document database (see FIG. 3) to securely store documents. Further, the document management module 214 may permit the automated transmission of documents including the user's medical information, identification documents, the emergency contact card, power of attorney documents, and the like.

In some embodiments, the emergency information module 216 provides a means for inputting and storing emergency contact information associated with the user's eContact Card (also referred to herein as the digital emergency contact card). The emergency information module 216 permits access to the user's Advanced Health Directive and Triage Medical information once the verification module 210 successfully verifies the EMT. In some embodiments, the emergency information module 216 allows the Deputy/Agent to access information associated with the emergency information module 216.

In some embodiments, the display module 218 is configured to display one or more graphic user interfaces, including, e.g., one or more user interfaces, one or more consumer interfaces, one or more video presenter interfaces, etc. In some embodiments, the display module 218 is configured to temporarily generate and display various pieces of information in response to one or more commands or operations. The various pieces of information or data generated and displayed may be transiently generated and displayed, and the displayed content in the display module 218 may be refreshed and replaced with different content upon the receipt of different commands or operations in some embodiments. In such embodiments, the various pieces of information generated and displayed in a display module 218 may not be persistently stored.

The display module 216 may be operable to automatically display patient-related information to the care provider and/or the EMT.

In some embodiments, the Deputy/Agent module 220 allows for the Deputy/Agent to interact with the system including receive messages and/or emails sent by the message module 202, or to access information provided by the emergency information module 216.

FIG. 3 illustrates a block diagram of the databases in operable communication with the computer system 100 and application program 200. A patient database 300 is operable to store patient-associated information including the patient's contact information, personal identifiable information (e.g., the patient's identification documents), medical information, and the like. The ER/Care Provider database 310 is operable to store care provider information including the care provider's identifiable information, contact information, care provider permissions and the like. The ER/Care Provider database 310 may also store information for the organization with which they are associated. The Deputy/Agent database 320 is operable to store deputy agent information including their contact information. The Deputy/Agent database 320 may also store a power of attorney, or other document or attestation which associated the deputy agent with a patient.

In some embodiments, the document management database 330 is operable to store various documents which are utilized by the verification module and documents module to verify the identities of users of the system. The document management database 330 may also store medical documents including medical records of the patient. Further, documents related to the deputy agent may also be stored.

In some embodiments, the emergency information database 340 stores information associated with the emergency contact cards and triage medical information. This information may be used to associate the emergency contact card with various users, such as by associating the emergency contact card with a patient, as well as by associating the patient with a deputy agent. The emergency information database 340 may also be operable to store information which allows for the association between a QR code and the patient's user profile.

In some embodiments, the Ledgerchain Vault database 350 provides secure storage and transactions of information within the system. The Ledgerchain Vault database 350 is a component of a secure document storage system which provides immutable storage. The Ledgerchain Vault database 350 secures, stores, and controls access to tokens, passwords, certificates and API keys.

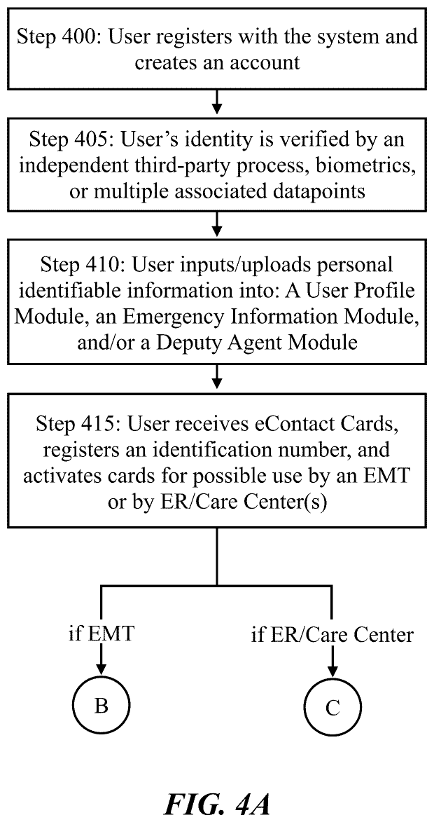

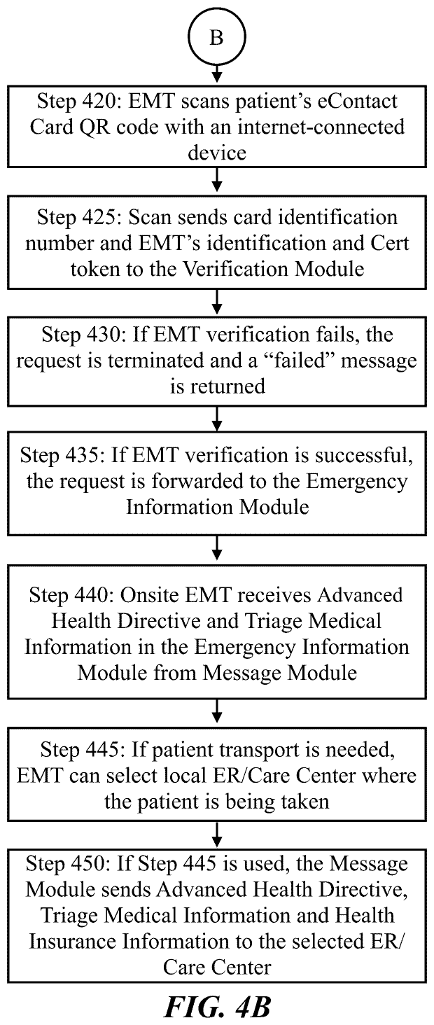

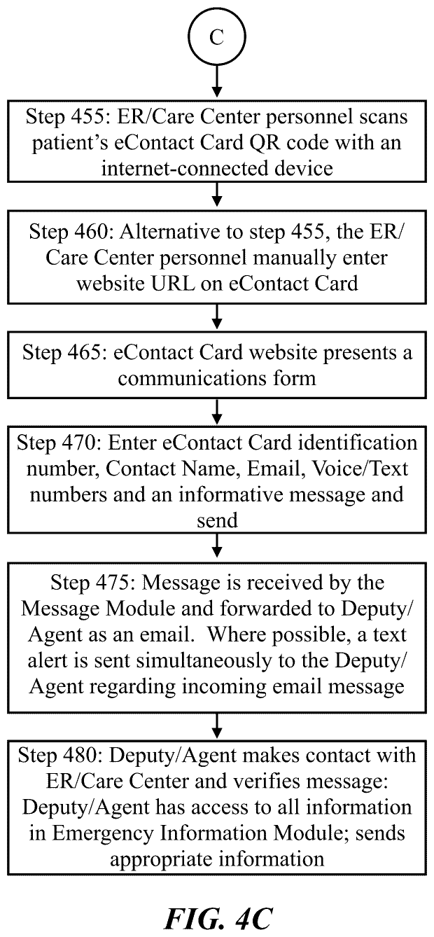

FIG. 4A illustrates a flowchart of a method for providing an emergency contact system for accessing medical and personal identifiable information. Specifically, FIG. 4A illustrates a process for the patient verification and emergency contact card generation processes. In step 400, the user registers with the system and creates an account. In step 405, the user's identity is verified by an independent third-party process, biometrics, or multiple associated datapoints. In step 410, the user inputs/uploads personal identifiable information into the user profile module, the emergency information module, and/or the deputy/agent module. In step 415, the user receives their eContact Cards and registers an identification number associated with the eContact Card. The user also activates the eContact Card for possible use by an EMT or by an ER/Care Center.

In reference to FIG. 4B, if the user is an EMT, the EMT scans the patient's eContact Card QR code with an internet-connected device (i.e., a computing device) in step 420. In step 425, the scan sends the card identification number and EMT's identification and Cert token to the verification module. In step 430, if the EMT verification fails, the request is terminated and a “failed” message is returned. In step 435, if the EMT verification is successful, the request is forwarded to the emergency information module. In step 440, the onsite EMT receives the advanced health directive and triage medical information in the emergency information module from the message module. In step 445, if patient transport is needed, the EMT can select a local ER/Care Center where the patient is being taken. In step 450, if step 445 is used, the message module sends the advanced health directive, triage medical information, and health insurance information to the selected ER/Care Center.

In reference to FIG. 4C, in step 455, the ER/Care Center personnel scans the patient's eContact Card QR code with an internet-connected device. Alternatively, the ER/Care Center personnel manually enter the website URL displayed on the eContact Card in step 460. In step 465, the eContact Card website presents a communications form. In step 470, the eContact Card identification number, contact names, email, voice/text numbers, and an information message is input and sent. In step 475, the message is received by the message module and forwarded to the Deputy/Agent as an email. Where possible, a text alert is transmitted to the Deputy/Agent regarding the incoming email message. In step 480, the Deputy/Agent makes contact with the ER/Care Center and verifies the message. The Deputy/Agent has access to all information contained in the emergency information module and the appropriate information is sent.

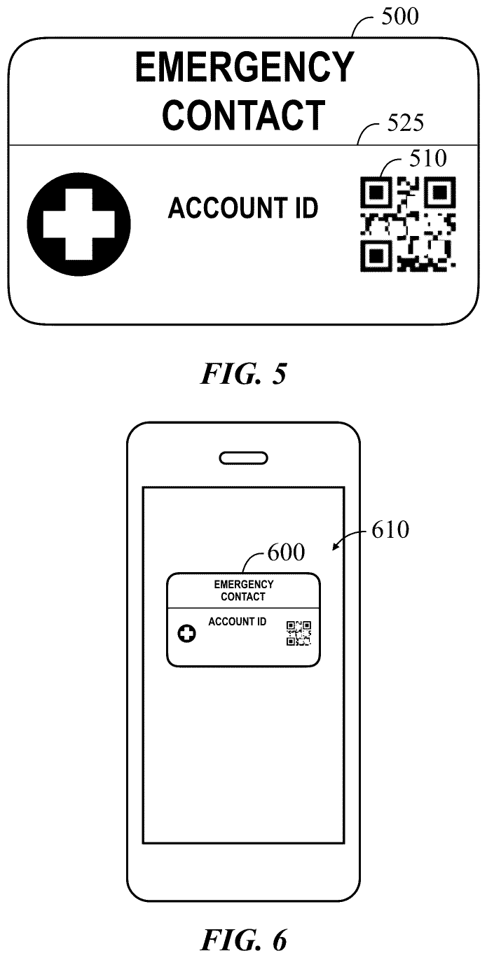

FIG. 5 illustrates the emergency contact card 500. The emergency contact card 500 may be a physical card which is carried by the patient. This allows care providers to scan the emergency contact card using a computing device (e.g., a smartphone or tablet). A QR code 510 may be printed on the emergency contact card 500 which allows the camera of the computing device to optically scan the QR code 510. The QR code 510 provides a unique code which is associated with the patient.

In some embodiments, the emergency contact card 500 includes an account identifier 530 displayed on the surface 525 of the emergency contact card 500. The account identifier can be optically scanned or manually typed into a prompt on the care provider's computing device to access the patient's information, as well as initiate the process described in FIG. 4 and further described in the embodiments provided in this disclosure. The emergency contact card 500 may also include the patient's name and other identifying information.

In some embodiments, the emergency contact card is provided as a digital emergency contact card 600 (see FIG. 6). The digital emergency contact card 600 is stored in the application programs emergency contact card database and provides a virtual means of transmitting information associated with the contact card as well as a means for accessing the patient's information if they do not possess a physical emergency contact card on their person. In such, the digital emergency contact card 600 may be presented to the care provider and scanned to provide access to the patient's information. This may be especially useful if the patient is not incapacitated, allowing them to use their personal computing device to show their emergency contact card to the care provider. The digital emergency contact card 600 may also include the QR code 510 allowing for the care provider to scan the emergency contact card user interface 610 illustrating the digital emergency contact card 600.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for the on-demand and automated transfer of medical information to on-site medical care providers, the system comprising:
at least one user computing device in operable connection with a network;
an application server in operable communication with the network, the application server configured to host an application program for the automated transfer of a plurality of patient-associated information, the application program having a user interface module for providing access to the medical information by a medical care provider on-site;
a digital emergency contact card including a scannable code to identify the patient and provide access to the plurality of patient-associated information, the QR code is associated with the patient-associated information to be displayed by the user interface module.

2. The system of claim 1, wherein the digital emergency contact card is also provided as a physical emergency contact card.

3. The system of claim 2, wherein the scannable code is a QR code displayed on a surface of the emergency contact card.

4. The system of claim 2, further comprising an account number displayed on a surface of the emergency contact card, wherein the account number enables manual entry of the account number into the interface module.

5. The system of claim 4, further comprising a website URL displayed on the surface of the emergency contact card.

6. The system of claim 1, further comprising a verification module to perform the step of verifying the identity of the care provider using the identifiable information they provide.

7. The system of claim 1, further comprising a message module to facilitate the transmission of at least one message to a deputy agent.

8. The system of claim 7, wherein the message module facilitates the transmission from the deputy agent of the patient-associated information to the ER/care center.

9. The system of claim 1, wherein a user module permits the association of a user account with a plurality of user permissions.

10. An emergency contact card to provide the on-demand and automated transfer of medical information to on-site medical care providers, comprising:
an emergency contact card including a scannable code displayed on a surface of the emergency contact card, the scannable code to provide selective access to a plurality of patient-associated information;
a care provider computing device to scan the scannable code;
a verification module to verify the identity of the on-site medical care provider;
a message module to automatically and selectively transmit the plurality of patient-associated information to the on-site medical care provider and care center personnel to transmit a message to a deputy agent associated with the patient.

11. The system of claim 10, wherein the emergency contact card is provided as a digital display of the emergency contact card.

12. The system of claim 10, wherein the scannable code is a QR code displayed on a surface of the emergency contact card.

13. The system of claim 10, further comprising an account identification number displayed on a surface of the emergency contact card, wherein the account number enables manual entry of the account number into the interface module.

14. The system of claim 10, wherein the verification module performs the following steps:
receiving an input from the user to indicate if the user is the on-site medical care provider; and
verifying the identity of the on-site medical care provider using the identifiable information they provide.

15. The system of claim 10, wherein the message module facilitates the automated transmission of the patient-associated information to the on-site medical care provider.

16. The system of claim 10, wherein a user module permits the association of a user account identification number with a plurality of user permissions.

17. A method for providing the on-demand and automated transfer of medical information to on-site medical care providers, the method comprising the steps of:
registering, via a user, to generate a user account, the user account generated via a user module;
selecting, via the user, a user-type to enable the user module to determine accessibility of Emergency Management Services personnel;
generating, via a document management module, an emergency contact card with an account identification number;
associating, via the user module, the emergency contact card with the user;
scanning, via an on-site medical care provider computing device, a scannable code provided with the emergency contact card;
transmitting, via a message module, an automated communication to an on-site medical care provider and
providing, via a verification module, access to a plurality of patient-associated information to the on-site medical care provider.
transmitting, via a communications module, a plurality of patient-associated information from emergency information module to ER/care center prior to patient arrival.

18. The method of claim 18, wherein the emergency contact card is provided as a digital emergency contact card on a user computing device.

19. The method of claim 18, wherein the scannable code is a QR code. The method of claim
